(19) United States
(12) Patent Application Publication
(10) Pub. No.: US 2014/0345628 A1
(43) Pub. Date: Nov. 27, 2014

(54) TEETH SEPARATING SYSTEM

(71) Applicant: Sherry Zhao, Tampa, FL (US)

(72) Inventor: Sherry Zhao, Tampa, FL (US)

(21) Appl. No.: 14/456,662

(22) Filed: Aug. 11, 2014

Related U.S. Application Data
(63) Continuation-in-part of application No. 13/200,469, filed on Sep. 23, 2011, now Pat. No. 8,800,568.
Application No. 13/200,469 is a continuation-in-part of application No. 12/930,905, filed on Jan. 19, 2011, now abandoned.

(51) Int. Cl. A61F 5/56

(52) U.S. Cl. CPC A61F 5/566; A61F 2005/563
USPC 128/861

(57) ABSTRACT

A left base component and a right base component with each base component having upper and lower surfaces. Each base component has an exterior edge, a central front point, laterally spaced rear points and an interior edge. Each base component has a rectangular central extent and arcuate forward extents and a semicircular rearward extent. The system also includes a support component in an arcuate configuration positionable in a generally vertical orientation. The support component has inner and outer faces. The support component has upper and lower edges. The upper edge is perpendicular to and integrally formed with the base component at the exterior periphery. The support component has terminal ends located adjacent to central regions of the rectangular central extents. The support component has an arcuate forward section between the base components. The lower surface of the base components is spaced from the upper surface of the forward section by an elevational space.

### RELATED APPLICATION

The present application is a continuation-in-part of pending application Sere. No. 13/200,469 filed Sep. 23/2011 which is a continuation-in-part of application Ser. No. 12/930,905 filed Jan. 19/2011, the subject matter of which applications ai incorporated herein by reference.

### BACKGROUND OF THE INVENTION

1. Field of the Invention

The present invention relates to a teeth separating system and more particularly pertains to abating tooth grinding through positioning between upper and lower teeth of a user, the abating being done in a safe, convenient and economical manner.

2. SUMMARY OF THE INVENTION

In view of the disadvantages inherent in the known types of teeth separating systems of known designs and configurations now present in the prior art, the present invention provides an improved teeth separating system. As such, the general purpose of the present invention, which will be described subsequently in greater detail, is to provide a new and improved teeth separating system and method which has all the advantages of the prior art and none of the disadvantages.

To attain this, the present invention essentially comprises a teeth separating system. The system includes a left base component and a right base component. Each base component has upper and lower surfaces. Each base component has an exterior edge, a central front point, laterally spaced rear points and an interior edge. Each base component has a rectangular central extent and arcuate forward extents and a semicircular rearward extent. The system also includes a support component in an arcuate configuration positionable in a generally vertical orientation. The support component has inner and outer faces. The support component has upper and lower edges. The upper edge is perpendicular to and integrally formed with the base component at the exterior periphery. The support component has terminal ends located adjacent to central regions of the rectangular central extents. The support component has an arcuate forward section between the base components. The lower surfaces of the base components is spaced from the upper surface of the forward section by an elevational space.

There has thus been outlined, rather broadly, the more important features of the invention in order that the detailed description thereof that follows may be better understood and in order that the present contribution to the art may be better appreciated. There are, of course, additional features of the invention that will be described hereinafter and which will form the subject matter of the claims attached.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of descriptions and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

It is therefore an object of the present invention to provide a new and improved teeth separating system which has all of the advantages of the prior art separating systems of known designs and configurations and none of the disadvantages.

It is another object of the present invention to provide a new and improved teeth separating system which may be easily and efficiently manufactured and marketed.

It is further object of the present invention to provide a new and improved teeth separating system which is of durable and reliable constructions.

An even further object of the present invention is to provide a new and improved teeth separating system which is susceptible of a low cost of manufacture with regard to both materials and labor, and which accordingly is then susceptible of low prices of sale to the consuming public, thereby making such teeth separating system economically available to the buying public.

Lastly, it is an object of the present invention to provide a new and improved teeth separating system for abating tooth grinding through positioning between upper and lower teeth of a user, the abating being done in a safe, convenient and economical manner,

These together with other objects of the invention, along with the various features of novelty which characterize the invention, are pointed out with particularity in the claims annexed to and forming a part of this disclosure. For a better understanding of the invention, its operating advantages and the specific objects attained by its uses, reference should be had to the accompanying drawings and descriptive matter in which there is illustrated preferred embodiments of the invention.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 is a perspective illustration of a teeth separating system constructed in accordance with the principles of the present invention.

FIG. 2 is a side elevational view of the system shown in FIG. 1.

FIG. 3 is a bottom view of the system shown in FIGS. 1 and 2.

The same reference numerals refer to the same parts throughout the various Figures.

### DESCRIPTION OF THE PREFERRED EMBODIMENT

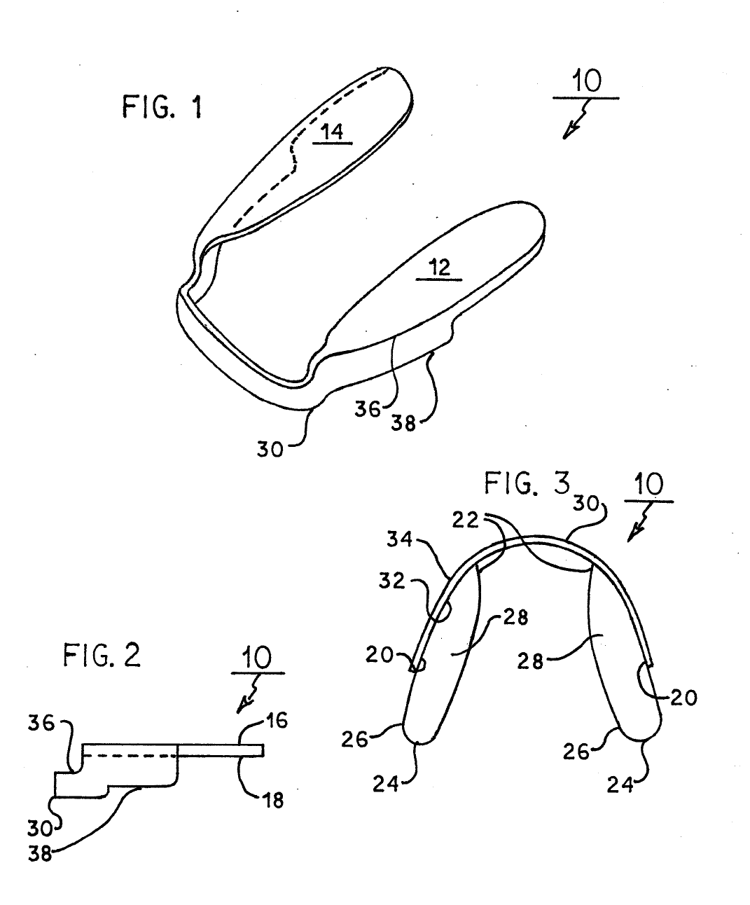

With reference now to the drawings, and in particular to FIG. 1 thereof, the preferred embodiment of the new and improved teeth separating system embodying the principles and concepts of the present invention and generally designated by the reference numeral 10 will be described.

The present invention is a teeth separating system 10 for abating tooth grinding through positioning between upper and lower teeth of a user. The abating is done in a safe, convenient and economical manner. The system includes a left base component 12 and a similarly configured right base component 14. The left base component and the right base component are positionable in a generally horizontal plane when in use between the teeth of a user while standing. Each base component has an upper surface 16 positionable in contact with the upper teeth of a user during use. Each base component has a lower surface 18 positionable in contact with the lower teeth of a user during use. The upper surface and lower surfaces are separated by a thickness of 3.0 millimeters plus or minus 10 percent throughout their entire extents. Each base component has an exterior edge 20 with a central front point 22 and laterally spaced rear points 24. The base component has an interior edge 26. Each base component has a rectangular central extent 28 with a length of 21 millimeters plus or minus 10 percent. Each base component has arcuate forward extents with radii of curvature of 36 millimeters plus or minus 10 percent. Each base component has a semicircular rearward extent with a radius of curvature of 5.5 millimeters plus or minus 10 percent.

The system also includes a support component 30 in an arcuate configuration. The support component is positionable in a generally vertical orientation when in use exterior of gums and interior of the cheeks of a user while standing. The support component has an inner face 32 and an outer face 34. The inner and outer faces are separated by a thickness of 1.5 millimeters plus or minus 10 percent throughout their entire extents. The support component has an upper edge 36 and a lower edge 38. The upper edge is perpendicular to and integrally formed with the base components at the exterior periphery. The support component has rearward sections with a height of 6 millimeters plus or minus 10 percent. The support component has terminal ends located adjacent to central regions of the rectangular sections. The support component has an arcuate forward section between the base components with a height of 4 millimeters plus or minus 10 percent and with a radius of curvature of 26.25 millimeters plus or minus 10 percent. The lower surface of the base components is spaced from the upper surface of the forward section by an elevational space of 4 millimeters plus or minus 10 percent. The support component between the arcuate forward sections and the rearward sections has a height of 4 millimeters plus or minus 10 percent. The support component is linear adjacent to the terminal ends and forming an angle of from 40 to 44 degrees. The support component has a midpoint spaced forwardly from the rearward extents of the base components by a distance of 51 millimeters plus or minus 10 percent.

The system in the primary embodiment is fabricated in one piece of a material with limited flexibility and elasticity. The material is chosen from the class of materials with limited flexibility and elasticity consisting of silicone and latex and plastic and organic materials.

As to the manner of usage and operation of the present invention, the same should be apparent from the above description. Accordingly, no further discussion relating to the manner of usage and operation will be provided.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

What is claimed as being new and desired to be protected by Letters Patent of the United States is as follows:

1. A teeth separating system comprising:
a left base component and a right base component, each base component having upper and lower surfaces, each base component having an exterior edge, a central front point, laterally spaced rear points, and an interior edge, each base component having a rectangular central extent and arcuate forward extents and a semicircular rearward extent; and
a support component in an arcuate configuration positionable in a generally vertical orientation, the support component having inner and outer faces, the support component having upper and lower edges, the upper edge being perpendicular to and integrally formed with the base components at the exterior periphery, the support component having terminal ends located adjacent to central regions of the rectangular central extents, the support component having an arcuate forward section between the base components, the lower surface of the base components being spaced from the upper surface of the forward section by an elevational space.

2. The system as set forth in claim 1 wherein the system is fabricated in one piece of a material with limited flexibility and elasticity, the material being chosen from the class of materials with limited flexibility and elasticity including silicone and latex and plastic and organic materials.

3. The system as set forth in claim 1 wherein the base components have a thickness of 3 millimeters plus or minus 10 percent and the support component has a thickness of 1.5 millimeters plus or minus 10 percent.

4. A teeth separating system (10) for abating tooth grinding through positioning between upper and lower teeth of a user, the abating being done in a safe, convenient and economical manner, the system comprising, in combination:
a left base component (12) and a similarly configured right base component (14), the left base component and the right base component being positionable in a generally horizontal plane when in use between the teeth of a user while standing, each base component having an upper surface (16) positionable in contact with the upper teeth of a user during use and a lower surface (18) positionable in contact with the lower teeth of a user during use, the upper surface and lower surfaces being separated by a thickness of 3.0 millimeters plus or minus 10 percent throughout their entire extents, each base component having an exterior edge (20) with a central front point (22) and laterally spaced rear points (24), each base component having an interior edge (26), each base component having a rectangular central extent (28) with a length of 21 millimeters plus or minus 10 percent, each base component having arcuate forward extents with radii of curvature of 36 millimeters plus or minus 10 percent, each base component having a semicircular rearward extent with a radius of curvature of 5.5 millimeters plus or minus 10 percent;
a support component (30) in an arcuate configuration, the support component being positionable in a generally vertical orientation when in use exterior of gums and interior of the cheeks of a user while standing, the support component having an inner face (32) and an outer face (34), the inner and outer faces being separated by a thickness of 1.5 millimeters plus or minus 10 percent throughout their entire extent, the support component having an upper edge (36) and a lower edge (38), the upper edge being perpendicular to and integrally formed with the base component at the exterior periphery, the support component having rearward sections with a height of 6 millimeters plus or minus 10 percent, the support component having terminal ends located adjacent to central regions of the rectangular sections, the support component having an arcuate forward section between the base components with a height of 4 millimeters plus or minus 10 percent and with a radius of curvature of 26.25 millimeters plus or minus 10 percent, the lower surfaces of the base being spaced from the upper surface of the forward section by an elevational space of 4 millimeters plus or minus 10 percent, the support component between arcuate forward section and the rearward sections having a height of 4 millimeters plus or minus 10 percent, the support component being linear adjacent to the terminal ends and forming an angle of from 40 to 44 degrees, the support component having a midpoint spaced forwardly from the rearward extents of the base components by a distance of 51 millimeters plus or minus 10 percent; and
the system being fabricated in one piece of a material with limited flexibility and elasticity, the material being chosen from the class of materials with limited flexibility and elasticity including silicone and latex and plastic and organic materials.
